(19) United States
(12) Patent Application Publication
(10) Pub. No.: US 2020/0072810 A1
(43) Pub. Date: Mar. 5, 2020

(54) DISSOLVABLE SENSOR SYSTEM FOR ENVIRONMENTAL PARAMETERS

(71) Applicant: KING ABDULLAH UNIVERSITY OF SCIENCE AND TECHNOLOGY, Thuwal (SA)

(72) Inventors: Muhammad M. HUSSAIN, Austin, TX (US); Kush MISHRA, Jaipur (IN); Aftab Mustansir HUSSAIN, Thuwal (SA); Meshaal Muhammed ABDULKAREEM, Thuwal (SA); Joanna NASSAR, Thuwal (SA)

(21) Appl. No.: 16/346,982

(22) PCT Filed: Nov. 21, 2017
(86) PCT No.: PCT/IB2017/057304

§ 371 Date: May 2, 2019

Related U.S. Application Data
(60) Provisional application No. 62/437,961, filed on Dec. 22, 2016.

(51) Int. Cl. G01N 33/00; A01G 25/16; G01N 27/30; B64C 39/02

(52) U.S. Cl. CPC G01N 33/0098; A01G 25/167; B64C 2201/126; B64C 39/024; G01N 27/308

(57) ABSTRACT

A sensor system includes at least one sensor configured to detect at least one environmental parameter, a processor coupled to the at least one sensor, and a dissolvable polymer encasing the sensor system.

### CROSS-REFERENCE TO RELATED APPLICATIONS

This application claims the benefit of priority of U.S. Provisional Patent Application No. 62/437,961 to Muhammad Mustafa HUSSAIN, et al., filed Dec. 22, 2016 and entitled “DRONE-BASED DATA COLLECTION FOR AUTOMATED PLANT MONITORING AND UP-KEEP,” the entire contents of which is incorporated herein by reference.

### BACKGROUND

### Technical Field

Embodiments of the subject matter disclosed herein generally relate to a dissolvable sensor system, a system including a plurality of dissolvable sensor systems, and method of making a dissolvable sensor.

Discussion of the Background

Agriculture consumes a significant amount of the Earth's freshwater with some studies concluding that agriculture consumes approximately 70% of the Earth's freshwater. Environmental changes have reduced the available amount of freshwater, and thus freshwater is quickly becoming a precious resource, which increases the overall costs of growing crops.

Conventional techniques for conserving water for growing crops involve monitoring water sensors placed in the soil around crops. These conventional sensors are typically expensive and provide limited information about the overall health of the crops. For example, these sensors provide generalized information about the moisture content of the soil, but do not indicate how much water is being used by any individual plant. This may result in some plants having access to sufficient quantities of water while other proximately-located plants not having access to sufficient quantities of water.

Further, moisture content of soil may not provide sufficient information about the growth of the plant themselves because the moisture content of soil is just one factor impacting crop growth. This can result in overwatering crops, which wastes precious water resources, or underwatering crops, which can result in crop destruction or producing crops that are undersized or have poorly formed shapes that do not correspond to the shapes consumers expect for a particular type of crop.

Accordingly, it would be desirable to provide methods and systems for more accurately monitoring various environmental parameters related to crop growth in a cost-effective manner.

### SUMMARY

According to an exemplary embodiment, there is a sensor system, which includes at least one sensor configured to detect at least one environmental parameter, a processor coupled to the at least one sensor, and a dissolvable polymer encasing the sensor system.

According to another embodiment, there is a system, which includes a plurality of sensor systems respectively configured to detect at least one environmental parameter of one of a plurality of plants, wherein each of the plurality of sensor systems is encased in a dissolvable polymer. The system also includes an unmanned aerial vehicle configured to collect a plurality of environmental parameters, which include the at least one environmental parameter of the plurality of plants, from the plurality of sensors. The system further includes a central system configured to receive the collected plurality of environmental parameters from the plurality of sensors from the unmanned aerial vehicle and to process the received plurality of environmental parameters.

According to a further embodiment, there is a method of making a dissolvable sensor. A sensor electrode is formed on a flexible thin-film substrate. A sensing film is deposited on the sensor electrode and the flexible thin-film substrate. The sensor electrode, the sensing film, and the flexible thin-film substrate are encased in a dissolvable polymer.

### BRIEF DESCRIPTION OF THE DRAWINGS

The accompanying drawings, which are incorporated in and constitute a part of the specification, illustrate one or more embodiments and, together with the description, explain these embodiments. In the drawings:

FIG. 1A is a schematic diagram of a sensor system according to an embodiment;

FIG. 1B is a schematic diagram of another sensor system according to an embodiment;

FIG. 2 is a schematic diagram of a sensor and a crop leaf according to an embodiment.

FIG. 3 illustrates a flowchart of a method for making a sensor used in a sensor system according to an embodiment;

FIGS. 4A-4D are schematic diagrams illustrating the production of a sensor used in a sensor system according to an embodiment;

FIGS. 5A and 5B are schematic diagrams of other sensor design according to embodiments;

FIG. 6 illustrates a flowchart of a method of using a sensor system according to an embodiment;

FIGS. 7A and 7B are schematic diagrams of a sensor system arranged on a crop according to embodiments;

FIG. 8 is a schematic diagram of a method of distribution of sensor systems onto crops in accordance with an embodiment;

FIGS. 9A and 9B are schematic diagrams of a method of collecting and processing sensor data according to an embodiment; and

FIG. 10 illustrates a schematic diagram of a central system according to an embodiment.

### DETAILED DESCRIPTION

The following description of the exemplary embodiments refers to the accompanying drawings. The same reference numbers in different drawings identify the same or similar elements. The following detailed description does not limit the invention. Instead, the scope of the invention is defined by the appended claims. The following embodiments are discussed, for simplicity, with regard to the terminology and structure of dissolvable sensor systems for monitoring environmental parameters related to crops. However, the embodiments to be discussed next are not limited to monitoring environmental parameters related to crops and the sensor systems can be employed for monitoring parameters for any use.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment a sensor system includes at least one sensor configured to detect environmental parameters, a processor coupled to the at least one sensor, and a dissolvable polymer encasing the sensor system.

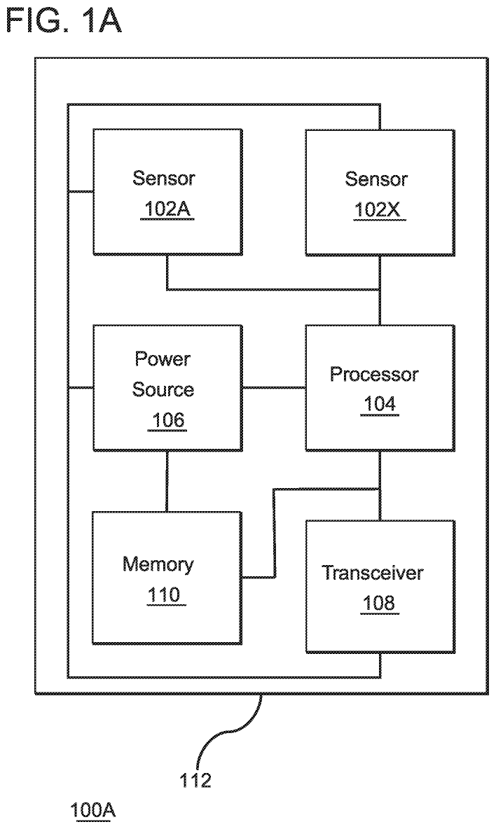

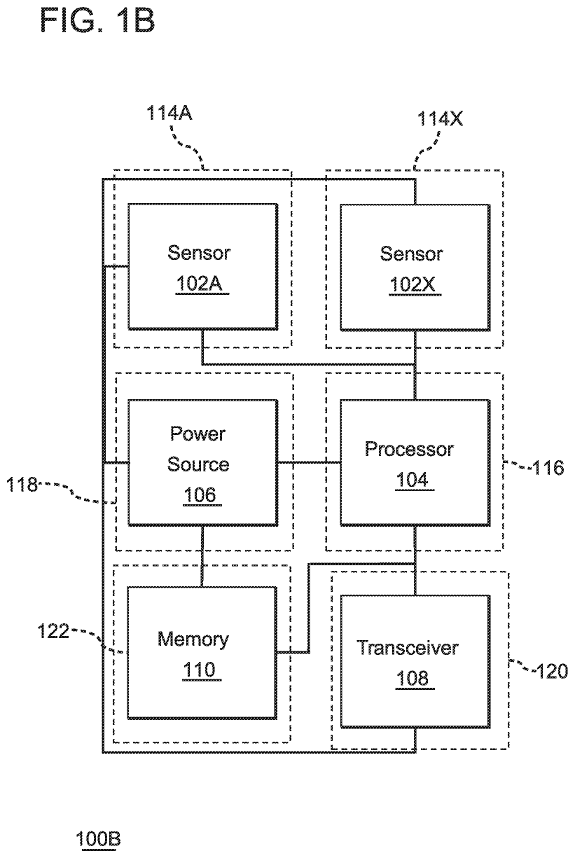

FIG. 1A is a schematic diagram of a sensor system according to an embodiment. The sensor system 100A is designed to be placed on crops and thus is configured to be as lightweight and thin as possible to avoid impacting crop growth. Further, the sensor system 100A is designed to operate in the same environment as the crops, but is also designed to dissolve in an environmentally friendly manner after a period of time so that harvested crops do not include the sensor system or any components of the sensor system.

The sensor system 100A includes one or more sensors 102A-102X coupled to a processor 104 and power source 106. A transceiver 108 and memory 110 are also both coupled to the processor 104 and power source 106. Further, the processor 104 is also coupled to the power source 106. All of these components are encased in a dissolvable polymer 112, which is permeable to gas. To minimize environmental impact when the dissolvable polymer encasing dissolves, components comprised of silicon, such as the processor, memory, and transceiver, are formed using thinned silicon so that these components disintegrate.

Depending upon design, the one or more sensors 102A-102X can be configured to monitor various environmental parameters, including temperature, pH, soil moisture content, air humidity, nutrient levels, pesticide levels, plant strain, plant growth, plant expansion, etc.

The processor 104 can be any type of processor, including a microprocessor, field programmable gate array (FPGA), application specific integrated circuit (ASIC), etc. Because the sensor system 100A is designed to be lightweight and powered by a source not directly connected to the power grid, the sensor system 100A benefits from using a simple, lightweight, and low-powered processor, such as an ASIC.

The power source 106 can be any type of power source, including a battery (e.g., a lithium ion battery), a solar array, a piezoelectric source generating power based on movement of the crop, etc. Transceiver 108 can be any type of transceiver using any type of wide-area network or local-area network wireless communication technology, including cellular technology, WFi technology, Bluetooth technology, etc. Using a local-area network wireless communication technology, such as WiFi or Bluetooth, provides the advantage of low power consumption by the transceiver 108. Memory 110 can be any type of memory and can store both program instructions for processor 104 and transceiver 108 (if applicable) and the parameters collected by the one or more sensors 102A-102X. Depending upon implementation, memory 110 can be a separate component or can be integrated in the processor 104.

FIG. 1B is a schematic diagram of a sensor system 100B according to another embodiment. In contrast to the sensor system of FIG. 1A, in which all of the components of the sensor system are commonly encased in the dissolvable polymer 112, the sensor system of FIG. 1B separately encases the various components in dissolvable polymer 114A-114X, 116, 118, 120, and 122. The separately encased components are externally connected with each other via conducting connections. The separate encasing of FIG. 1B allows different components of the sensor system to be placed on different parts of the crops, or even some on the crops and some on the surrounding soil. Thus, for example, the one or more sensors 102A-102X can be placed directly on the leaf of crops and the other components can be placed on the stems of crops or even on or in the soil. Further, some of the one or more sensors 102A-102X can be placed on a crop and others of the one or more sensors 102A-102X can be placed in the soil to monitor characteristics of the soil.

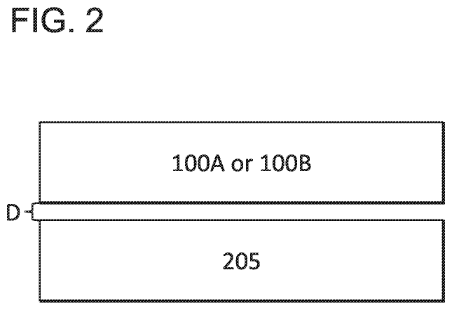

In an embodiment, the sensor and/or sensor system is configured so that it adheres to crops due to van der Waals force, which is a well-known force from physical chemistry arising from distance dependent interactions between atoms and is the force commonly believed to be the reason certain animals, such as Geckos, can stick to walls and ceilings. Adherence due to van der Waals force will be described in connection with FIG. 2, which illustrates a sensor system 100A or 100B separated by a distance D from, for example, a crop leaf 205. It will be recognized that although the sensor system 100A and 100B and the crop leaf 205 will be touching each other, there will be a distance D between the two objects due to the respective surface roughness of the two objects. Accordingly, the non-retarded van der Waal's free energy per unit area W between flat surfaces of the sensor system 100A or 100B and the crop leaf 205 separated by a distance D is:

$$W = - \frac{A}{12 π {D}^{2}}$$

where A is the Hamaker constant, having values depending on the atomic density of the flat surfaces of sensor system 100A or 100B and crop leaf 205. The attractive force per unit area between the flat surfaces of sensor system 100A or 100B and crop leaf 205 is:

$$F = \frac{δ W}{δ D} = \frac{A}{6 π {D}^{3}}$$

Assuming a Hamaker constant A of 10⁻¹⁹ Joules and a distance D of 100 nm, the force per unit area is approximately 5 N/m². Accordingly, a van der Waals force can suspend the sensor system 100A or 100B to the crop leaf 205 against gravity when the sensor system 100A or 100B weighs 50 mg and has a surface area larger than 1 cm×1 cm.

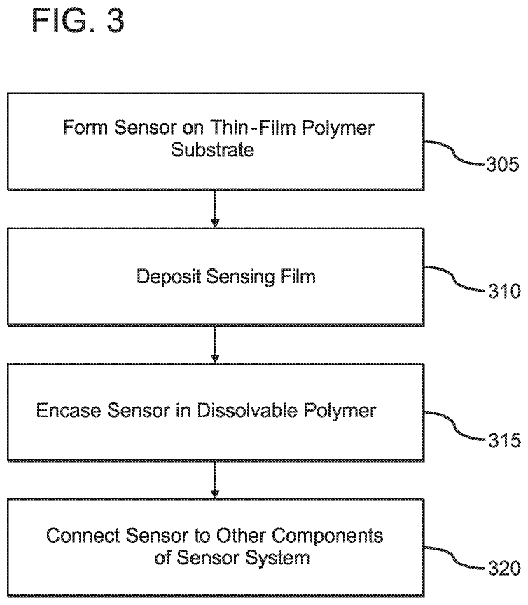

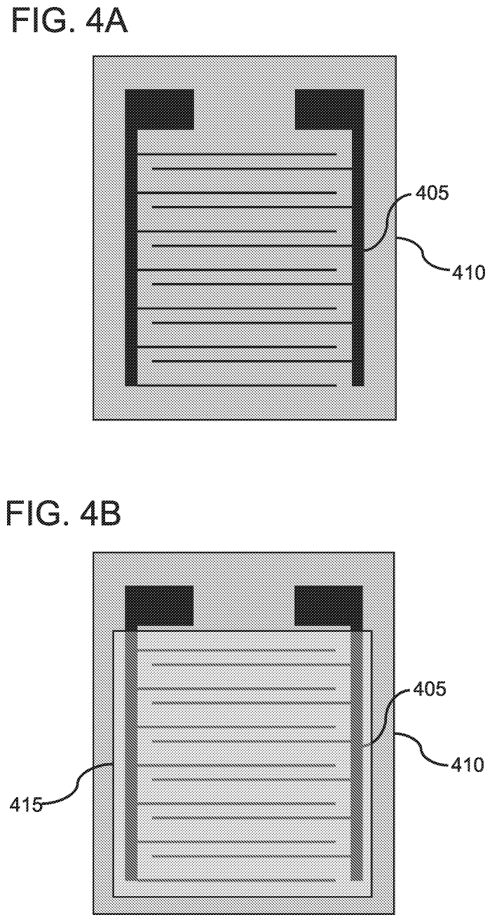

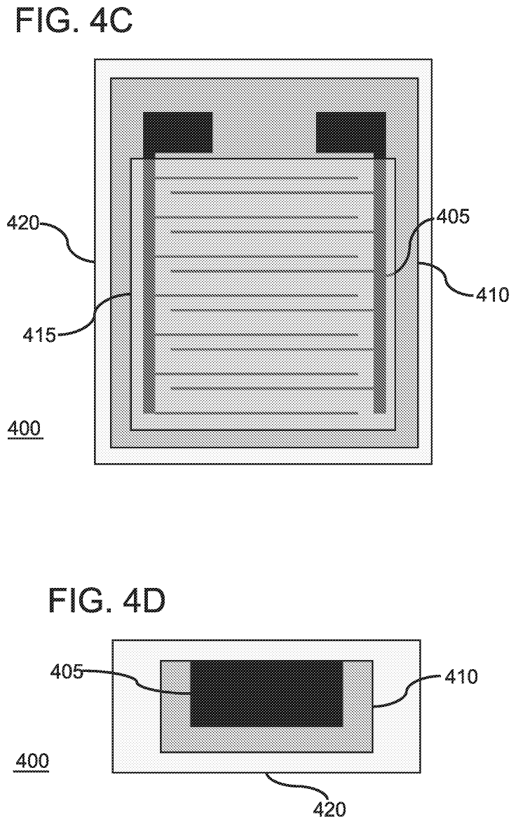

FIG. 3 illustrates a flowchart of a method for making a sensor used in a sensor system according to an embodiment, which will be described in connection with FIGS. 4A-4D. As illustrated in FIG. 4A, a sensor electrode 405 is formed on a flexible substrate 410 (step 305). The flexible substrate 410 can be a polymer substrate, such as a polyimide substrate. The sensor electrode 405 can be composed of any type of metal or other electrically conductive material, such as titanium/gold (Ti/Au), platinum (Pt), or even silver (Ag). The conductive pattern of the sensor electrode 405 can be designed to be extremely thin, e.g., approximately 50 nm, so that the sensor itself can disintegrate and be washed away prior to packaging of the particular crop. In an embodiment, the thickness of the sensor electrode 405 is less than 100 nm (i.e., in the nanomaterial regime) so that the dissolution of the sensor can be achieved in a timely manner. Due to the thinness of the conductive material of the sensor electrode 405 and the use of dissolvable metals, the sensor can fully disintegrate in water after a period of time, making the sensor environmentally inert.

As illustrated in FIG. 4B, sensing film 415 is formed on top of the sensing electrode 405 (step 310). The sensing film 415 can be comprised of, for example, dissolvable oxides, metal oxides, polymers (e.g., polyimide), graphene oxide, titanium dioxide, etc. The particular type of sensing film depends upon the desired environmental parameters to be sensed. Thus, depending upon the type of sensing film, the sensor can sense humidity/moisture levels, pH level of the soil, O₂ and/or CO₂ concentrations, nitrates concentrations, phosphorus concentrations, as well as other important gases that reflect biological activity and allow monitoring of soil quality for optimized healthy crop growth.

CMOS processing systems can be employed to fabricate the sensor electrode 405 and sensing film 415 on the flexible substrate 410. Alternatively, the sensor electrode 405 and sensing film 415 can be formed using a roll-to-roll fabrication technique in which the sensor electrodes 405 and the sensing film are respectively formed on rolls and then individually applied to another substrate roll, which is subsequently cut into individual sensors.

As illustrated in FIGS. 4C and 4D, the sensor electrode 405, flexible substrate 410, and sensing film 415 are then encased in a dissolvable polymer 420 (step 315) to form a dissolvable sensor 400. In an embodiment, the dissolvable polymer can be polyvinyl alcohol (PVA), a water-soluble synthetic polymer, a polyimide, etc. The thickness of the dissolvable polymer 420 is selected to correspond to how long the sensor is intended to operate before dissolving due to expected environmental factors. In the case of a polyimide, a 4 μm thin film of the polymer disintegrates in approximately 2-3 months when exposed to a saline solution.

It should be recognized that the illustration in FIG. 4D, which is a schematic cross-sectional side-view, of the sensor 400 is for purposes of explanation and that the sensor 400, including the encasing polymer 420, need not have a rectangular cross-section but can have any shaped cross-section. Although the sensing film 415 is not illustrated in the cross-sectional view of FIG. 4D, it will be arranged on top of the sensing electrode 405 and flexible substrate 410 as described above. Finally, the sensor electrode 405 is connected to other components of the sensor system (step 320).

Although the description above involves the production of a sensor, the description equally applies to the production of a sensor system, in which case step 320 would occur prior to the dissolvable polymer encasing step 315 so that the components before being commonly or separately encased in the dissolvable polymer.

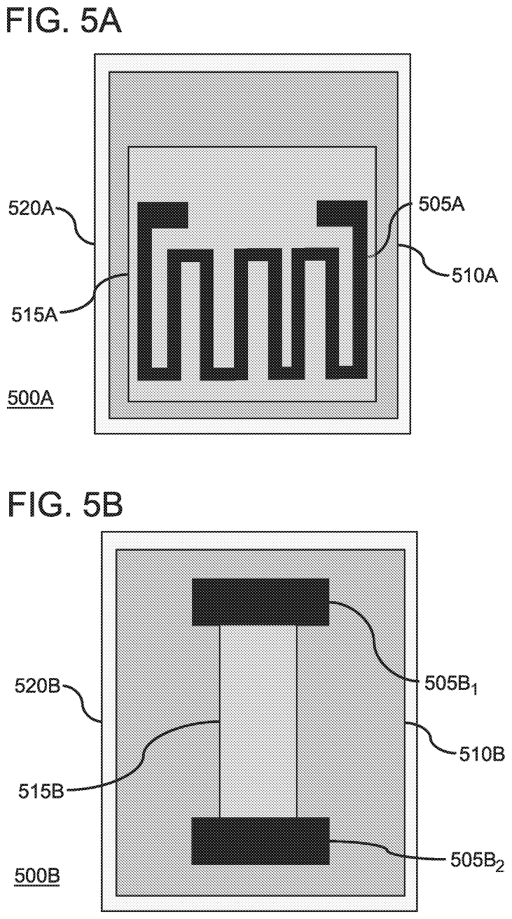

The particular sensor design illustrated in FIGS. 4A-4D is merely exemplary and other sensor designs can be employed, such as those illustrated in FIGS. 5A and 5B. The sensor 500A in FIG. 5A includes a sensing film 515A on top of the sensor electrode 505A and the flexible substrate 510A, all of which is encapsulated in a dissolvable polymer 520A. The sensor electrode 505A has a resistive temperature detector (RTD) structure comprising a dissolvable metal conductor, semi-conductive material, or an insulator (e.g., a metal oxide), and is designed to detect local temperature and heat generated in the vicinity of the sensor 500A (e.g., the temperature of and heat generated by a crop). In contrast to the interdigitated sensor electrode 405 in FIGS. 4A-4D, the sensor electrode 500A in FIG. 5A has a serpentine shape.

The sensor illustrated in FIG. 5B can be used to monitor salinity (i.e., salt concentration) in, for example, soil. The sensor 500B includes a sensing film 515B coupled between first 505B₁ and second 505B₂ electrodes, both of which are on flexible substrate 510B. The sensing film 515B can be an insulator-like polyimide, a metal oxide, and/or a semiconducting material. The first 505B₁ and second 505B₂ electrodes, flexible substrate 510B, and sensing film 515B are encapsulated in a dissolvable polymer 520B. Sensor 500B monitors salinity based on changes in the resistivity/conductivity of the dissolvable polymer 520B.

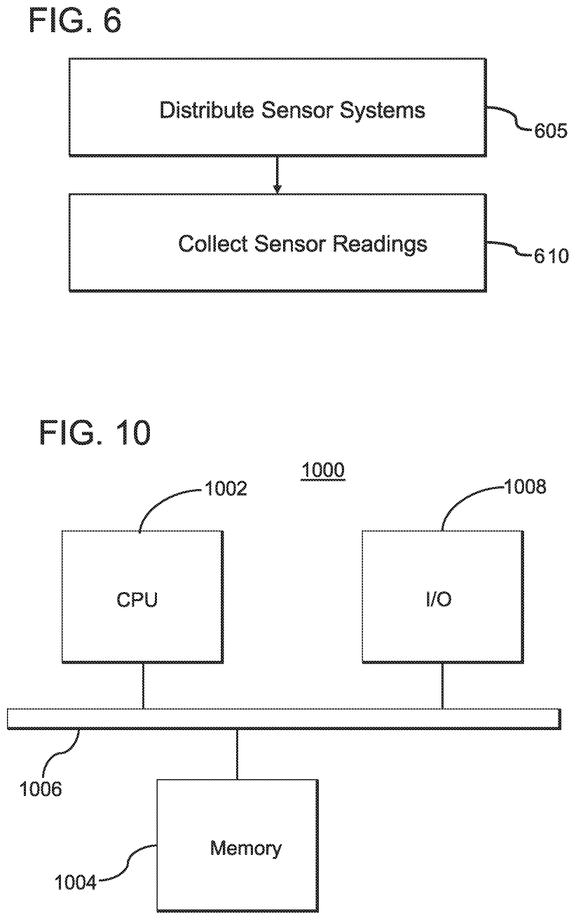

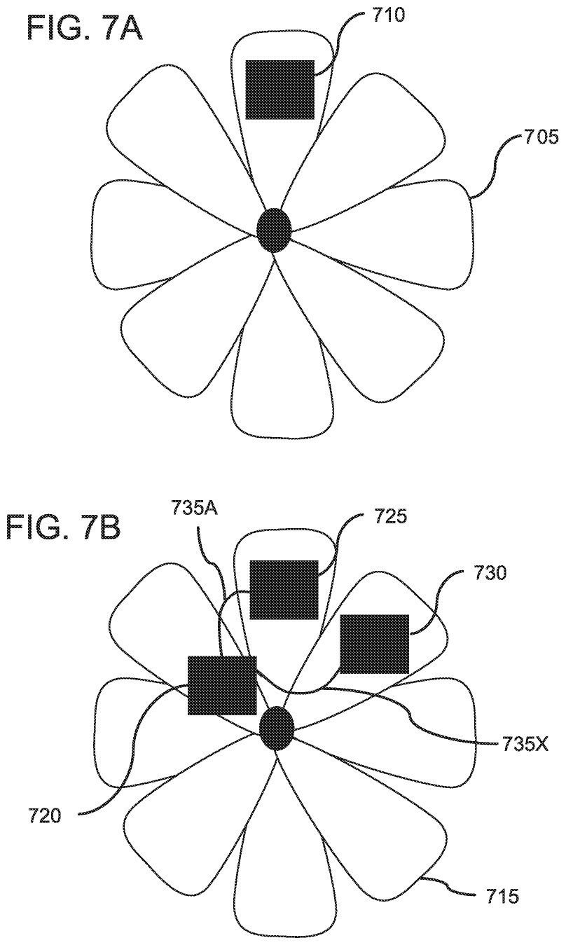

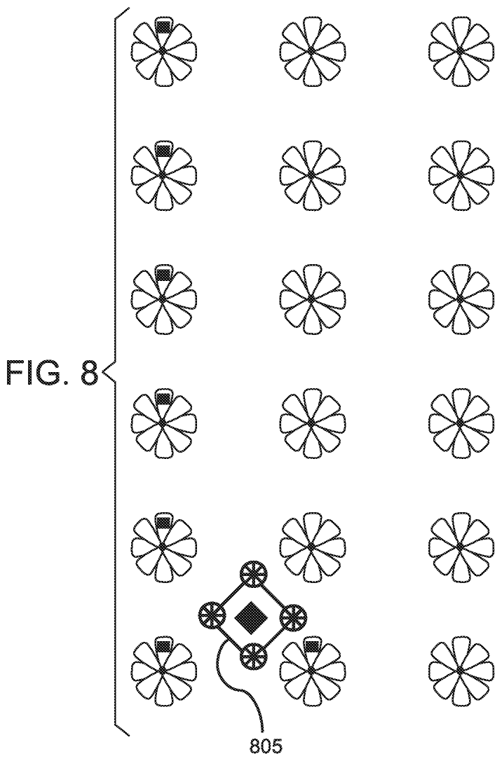

Turning to FIG. 6, now that one or more sensor systems have been produced, the sensor systems can be distributed, i.e., applied to crops (step 605) and then readings of the sensors can be collected (step 610). FIGS. 7A and 7B respectively illustrate a commonly encapsulated sensor system and a separately encapsulated sensor system applied to a crop. Specifically, as illustrated in FIG. 7A, a commonly encapsulated sensor system 710 is applied to a leaf of crop 705. As illustrated in FIG. 7B, a sensor system comprising separately encapsulated components 720, 725, and 730 are applied to different leaves of crop 715. The components are conductively coupled by one or more conductors 735A-735X. Although FIGS. 7A and 7B illustrate the sensor system being applied to leaves of crops, the sensor system can be applied to other parts of crops and can be applied to the soil itself.

FIG. 8 is a schematic diagram of a method of distribution of sensor systems onto crops according to an embodiment. In the illustrated method, an unmanned aerial vehicle, such as a drone 805, is used to distribute the sensor systems. Specifically, in FIG. 8 the drone 805 has already applied sensor systems to a first row of crops and to a first crop in a second row of crops, and the drone 805 is moving to the second crop in the second row for application of the sensor system. The drone will continue to apply the sensor systems to the remaining crops. The drone can apply the sensor systems to individual crops by directly, physically placing the sensor system on a particular part or parts of the crop or can drop the sensor system from above the crop and allow the sensor system to fall due to gravity onto the crop and then naturally adhere to the crop. Although FIG. 8 illustrates a distribution of sensor systems on each crop, sensor systems can be placed on less than all crops, such as by applying one sensor system to one crop that is part of a number of proximately located crops.

The proximity of crops for being proximately located is determined based on how well environmental parameters for one crop corresponds to those of other crops. This may vary based on the particular environmental parameter being detected. For example, temperature and air humidity are parameters that should be similar for crops over a relatively large area (assuming the crops are being grown on a relatively flat surface subject to relatively similar amounts of light), whereas pH and soil moisture content can vary enough that only crops that are located very close together can be subject to the use of a common sensor system. Thus, sensor systems having different components can be distributed to different crops so that one crop may include multiple sensors and crops considered to be proximately located can have fewer sensors that may or may not duplicate the sensors of the one crop. This provides a cost-savings advantage because it allows the use of sensor systems that do not contain sensors that would provide environmental parameters that are similar to those of other sensors.

Distributing sensor systems using a drone as described in connection with FIG. 8 is one of many ways to distribute the sensor systems and the sensor systems can be distributed using other mechanisms, such as being applied by hand. The advantage of using an automated method, such as a drone, is that it reduces the costs of the sensor system distribution.

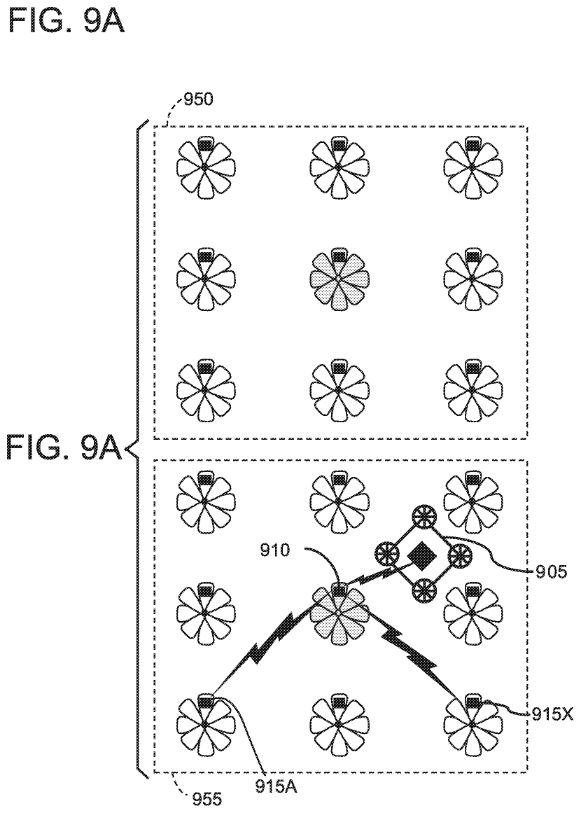

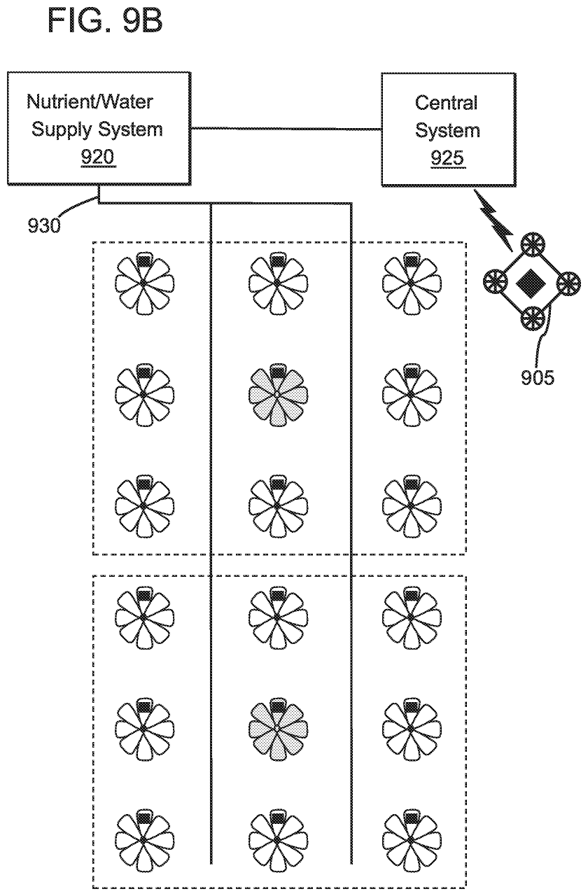

The collection of sensor readings can also be performed using an unmanned aerial vehicle, such a drone, an example of which is illustrated in FIGS. 9A and 9B. This example employs a zoned sensor collection in which crops are divided into separate zones 950 and 955. One sensor system within each zone is designated as the collection node, which in this example is sensor system 910. Each sensor system 915A-915X within the zone 955 (only two sensor systems are labeled for ease of illustration) provides measured environmental parameters to the collection sensor system 910. The collection node sensor 910 then communicates its own collected environmental parameters, as well as those collected by other sensor systems within the zone 955, to drone 905.

Turning now to FIG. 9B, depending upon the type of communications components carried by drone 905, the drone 905 can then either communicate the collected environmental parameters to a central system 925 via a wide area network, e.g., a cellular network, or the drone 905 can wait until it returns to the central system 925 to provide the collected environmental parameters to a storage and processing system. In the latter case the drone 905 can have a wired or wireless connection to the collection station to convey the collected environmental parameters. The central system 925 (details of which will be described in connection with FIG. 10) processes the collected environmental parameters and provides control instructions to a nutrient/water supply system 920 via communication connection, which can be a wired or wireless connection. The nutrient/water supply system 920 then supplies nutrients and/or water via distribution system 930 to one or more of the plants. The distribution system 930 is configured so that the amount of nutrients and/or water can be supplied on a per plant basis so that each plant receives enough nutrients and/or water without providing excess nutrients and/or water. The distribution system 930 can also be configured so that the amount of nutrients and/or water is supplied to a group of plants that are subject to the same environmental parameters.

The zoned collection system is advantageous because the sensor systems 915A-915X within a zone can employ very low power for conveying the measured environmental parameters to the sensor system 910 acting as the collection node and this sensor system can then employ higher power to convey the collected environmental parameters to the drone 905. If this is the case, the collection node 910 can have a larger power source for being able to maintain the communication with the other sensors. Of course, depending upon configuration of the crops relative to each other, the drone 905 can be configured to fly very close to each crop to achieve the same low power communications achieved by the zoned system.

The zoned collection systems illustrated in FIGS. 9A and 9B are merely exemplary and other types of collection systems can be employed. For example, environmental parameters can be collected using a so-called “matrix” technique in which a crop communicates its measured environmental parameters to a sensor system for a second crop, which then communicates those parameters and the second crop's measured environmental parameters to a sensor system for a third crop. This process repeats until an end node is reached from which the set of collected environmental parameters are communicated to a drone or to a fixed-location collection device. In one application, each sensor system directly communicates with the drone when the drone is passing by.

Regardless of the particular collection technique, the sensor systems can be configured to flush their local memories of the stored environmental parameters after the parameters are forwarded to another node or collected by the drone. This is particularly advantageous because it minimizes the amount of memory required by the sensor systems, which reduces the overall size and cost of the memory.

The sensor systems can be programed to collect environmental parameters from their respective sensor(s) at preprogrammed intervals and can also be programmed to forward the collected environmental parameters to a central node (when using a zoned collection technique) or another sensor system (when using a matrix collection technique) at preprogrammed interviews. Further, at certain intervals the drone will receive the collected environmental parameters and forward them to an environmental parameter collection, storage, and processing system.

FIG. 10 is a schematic diagram of central system according to an embodiment. The environmental parameter collection, storage, and processing system 1000 may include a processor 1002 and a storage device 1004 that communicate via a bus 1006. An input/output interface 1008 also communicates with the bus 1006 and allows an operator to communicate with the processor or the memory, for example, to input software instructions for operating the sensing system. The computing device 1000 may be a controller, a computer, a server, etc.

The environmental parameter collection, storage, and processing system 1000 can process the collected environmental parameters and provide this information, either all of the information or in a summary form, to an output (e.g., printer, display, etc.) via input/output interface 1008. Further, the system 1000 can output recommendations, such as areas requiring more or less water, areas requiring changes to the soil pH, locations of potential crop disease, etc. The system 1000 can also be connected to other systems so that it can control the other systems based on the collected and analyzed environmental parameters, such as controlling the amount of water, fertilizer, etc. applied to different crops or different groups of groups.

The use of the disclosed dissolvable sensor systems allows for more controlled use of water and fertilizer. This is particularly advantageous for semi-arid environments because irrigation is performed based on the actual requirements of a particular individual crop instead of a generalized indication of the soil moisture content across a large number of crops. Further, the cost for measuring and supplying essential nutrients can be reduced because these can be applied to the particular individual crops that actually need the nutrients instead of simply spraying nutrients across an entire field of crops.

Collection of environmental parameters on a per plant basis or for a set of proximately-located plants having similar environmental parameters also allows for more control over the specific flavor and nutrients of the crop by controlling the nutrient levels, water levels, amount of pesticides, ect. for each individual plant.

The ability to use low-power communications for environmental parameter collection is particularly advantageous because it allows use of a smaller power source, and thus reduces the surface area of the crops that may be occupied and/or obscured by the power source.

The dissolvable sensor systems can be used in a variety of different applications. The collected environmental parameters can be shared with metrological and other agencies for better forecasting. The dissolvable sensor systems can also be employed in large scale industrial applications to automate crop field data collection and more precise control of provision of water, nutrients, pesticides, etc. The low-cost of the dissolvable sensor systems is particularly advantageous for addressing crop production in poor and developing nations. The information about individual plants and groups of plants collected from the dissolvable sensor systems can be used in research and development into the making of plants.

The disclosed embodiments provide a dissolvable sensor system used for monitoring crops. It should be understood that this description is not intended to limit the invention. On the contrary, the exemplary embodiments are intended to cover alternatives, modifications and equivalents, which are included in the spirit and scope of the invention as defined by the appended claims. Further, in the detailed description of the exemplary embodiments, numerous specific details are set forth in order to provide a comprehensive understanding of the claimed invention. However, one skilled in the art would understand that various embodiments may be practiced without such specific details.

Although the features and elements of the present exemplary embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

1. A sensor system, comprising:
at least one sensor configured to detect at least one environmental parameter;
a processor coupled to the at least one sensor; and
a dissolvable polymer encasing the sensor system.

2. The sensor system of claim 1, wherein a thickness of the dissolvable polymer encasing the sensor system has a predetermined thickness corresponding to a time period in which the dissolvable polymer disintegrates when exposed to an environment.

3. The sensor system of claim 1, wherein the at least environmental parameter is at least one of soil moisture content, temperature, humidity, nutrient levels, pesticide levels, plant expansion, plant strain, and plant growth.

4. The sensor system of claim 1, wherein the sensor system has a predetermined weight and surface area so that the sensor system is attached to a plant surface by van der Waals force.

5. The sensor system of claim 1, further comprising:
a battery or solar array coupled to the at least one sensor and the processor to provide power.

6. The sensor system of claim 1, wherein the at least one sensor includes a first sensor and a second sensor, wherein the first and second sensors are configured to detect different environmental parameters.

7. The sensor system of claim 1, further comprising:
a communication transceiver coupled to the processor.

8. The sensor system of claim 1, wherein the at least one sensor and processor are commonly encased in the dissolvable polymer.

9. The sensor system of claim 1, wherein the at least one sensor and processor are separately encased in the dissolvable polymer and the separately encased at least one sensor and processor are electrically coupled to each other.

10. The sensor system of claim 1, wherein the at least one sensor includes a sensing film on top of a sensing electrode, which is on top of a flexible substrate.

11. The sensor system of clam 10, wherein the sensor electrode is composed of dissolvable metal.

12. The sensor system of clam 10, wherein the sensing film is composed of a dissolvable oxide, metal oxide, polymer, graphene oxide, or titanium oxide.

13. A system, comprising:
a plurality of sensor systems respectively configured to detect at least one environmental parameter of one of a plurality of plants, wherein each of the plurality of sensor systems is encased in a dissolvable polymer;
an unmanned aerial vehicle configured to collect a plurality of environmental parameters, which include the at least one environmental parameter of the plurality of plants, from the plurality of sensors; and
a central system configured to receive the collected plurality of environmental parameters from the plurality of sensors from the unmanned aerial vehicle and to process the received plurality of environmental parameters.

14. The system of claim 13, further comprising:
a nutrient and/or water supply system communicatively coupled to the central system and configured to control supply of nutrients and/or water to the plurality of plants based on the processed plurality of environmental parameters.

15. The system of claim 13, wherein the at least environmental parameter is at least one of soil moisture content, temperature, humidity, nutrient levels, pesticide levels, plant expansion, plant strain, and plant growth.

16. A method of making a dissolvable sensor, the method comprising:
forming a sensor electrode on a flexible thin-film substrate;
depositing a sensing film on the sensor electrode and the flexible thin-film substrate; and
encasing the sensor electrode, the sensing film, and the flexible thin-film substrate in a dissolvable polymer.

17. The method of claim 16, wherein the sensor electrode and the sensing film are formed on the flexible thin-film substrate using CMOS processing.

18. The method of claim 16, wherein the sensor electrode and sensing film are formed on the thin-film polymer substrate using roll-to-roll fabrication.

19. The method of claim 16, further comprising:
connecting the sensor electrode to a component of a sensor system.

20. The method of claim 16, wherein the sensor electrode is composed of a dissolvable metal and the sensing film is composed of a dissolvable oxide, metal oxide, polymer, graphene oxide, or titanium oxide.
